(19) United States
(12) Patent Application Publication
(10) Pub. No.: US 2005/0276794 A1
(43) Pub. Date: Dec. 15, 2005

(54) Composition and method for improving pancreatic islet cell survival

(76) Inventors: Klearchos K. Papas, Maple Grove, MN (US); Andreas M. Papas, Kingsport, TN (US); Konstantinos A. Papas, Jonesborough, TN (US)

Correspondence Address: DONNA J. RUSSELL, 1492 ANTHONY WAY, MT. JULIET, TN, 37122, US

(21) Appl. No.: 11/148,657

(22) Filed: Jun. 9, 2005

Related U.S. Application Data
(60) Provisional application No. 60/578,210, filed on Jun. 9, 2004.
(60) Provisional application No. 60/591,558, filed on Jul. 27, 2004.

(51) Int. Cl. A61K 45/00; C12N 5/08

(52) U.S. Cl. 424/93.7; 435/366

(57) ABSTRACT

The invention provides a composition for protecting islet cells during isolation and transplantation, as well as a method for increasing survival of islets and islet cells during harvest from the donor pancreas, isolation and culture, transportation, and transplantation into the recipient. The composition provides at least one Vitamin E homolog that, when combined with cell culture media, increases islet cell survival.

### CROSS-REFERENCE TO RELATED APPLICATIONS

This application claims the benefit of earlier-filed U.S. Provisional Patent Applications Nos. 60/578,210 filed 9 Jun. 2004 and 60/591,558 filed 27 Jul. 2004.

### FIELD OF THE INVENTION

The present invention relates to compositions and methods for increasing cell survival during and post-isolation from a tissue donor, and methods for increasing cell survival post-transplantation.

### BACKGROUND OF THE INVENTION

About 800,000 people in the United States now have type 1 diabetes, and about 30,000 people develop it each year. According to the Juvenile Diabetes Research Foundation, the only significant therapy for people who have already developed the disease is to replace the destroyed beta cells or the beta cell function. Pancreas transplants may restore insulin production, but because of significant risks the procedure has been limited, and is done primarily in recipients who are also undergoing kidney transplantation.

Following the development of the Edmonton Protocol (N. Engl. J. Med. (2000) 343: 230-8), intraportal pancreatic islet cell transplantation became a therapeutic choice for normalizing blood glucose regulation in patients with type 1 diabetes. Long-term correction of type 1 diabetes by intraportal islet transplantation, however, has been achieved in only a fraction of islet transplant recipients. Generally, studies have demonstrated that approximately 10,000 islet equivalents (IEqs) per kilogram of body weight are necessary to produce insulin independence for any period of time, requiring islets from at least two donor pancreata for most transplant procedures. Only about 3,000 pancreata become available each year, however, so much effort has been focused on methods for utilizing fewer islet cells per transplant procedure.

In a transplant procedure such as that described in the Edmonton Protocol, the donor pancreas is removed and the pancreatic ducts are perfused in a controlled fashion with cold enzyme. The islets are separated by mechanical dissociation, and then purified for transplant using continuous gradients of Ficoll-diatrizoic acid in an apheresis system. Once an islet suspension has been prepared, it is infused into the hepatic portal vein of the recipient. Within a few days after this procedure, however, studies have indicated that at least half of the transplanted beta (islet) cells have undergone apoptotic cell death triggered by hypoxic and chemokine/cytokine-mediated stress. If significant numbers of these cells could be retained, fewer numbers of cells would be necessary for each procedure and insulin regulation woud be more easily achieved in each patient.

Culture media in current use provide antioxidants and may provide certain forms of Vitamin E (e.g., alpha-tocopherol and/or Trolox, a synthetic analog of a-tocopherol) but do not typically provide formulations that promote sufficient levels of islet cell survival to decrease the number of cells that must be harvested and transplanted in order to produce the desired outcome.

What is needed are compositions and methods for increasing islet cell survival during the harvesting and transplantation process.

### SUMMARY OF THE INVENTION

The present invention relates to a composition comprising at least one Vitamin E homolog such as an effective amount of alpha-tocopherol, gamma-tocopherol, or a combination of Vitamin E homologs such as alpha- and gamma-tocopherol in combination with a cell transfer or cell culture medium. The invention also relates to a method for increasing pancreatic islet beta cell survival during harvest, pre-transplant and post-transplant into a recipient subject, the method comprising contacting the islet cells with an aqueous solution of alpha-tocopherol, gamma-tocopherol, or a combination of alpha- and gamma-tocopherol. In one embodiment, a tocopherol homolog is admixed with a cell culture medium. The tocopherol homolog may be admixed with the medium before the islet cells are introduced into the medium, after the islet cells are introduced into the medium, or simultaneously as the islet cells are introduced into the medium.

### BRIEF DESCRIPTION OF THE DRAWINGS

FIG. 1 through FIG. 7 are photographs taken under microscopy of culture of hand-picked islet cells for the indicated number of hours in the presence of the following nutrients added to the culture medium: alpha-tocopherol (FIG. 1); gamma-tocopherol (FIG. 2); alpha-tocopheryl succinate (FIG. 3); alpha-tocopherol and gamma-tocopherol (FIG. 4); alpha-tocopherol and alpha-tocopheryl succinate (FIG. 5); gamma-tocopherol and alpha-tocopheryl succinate (FIG. 6); and alpha-tocopherol, gamma-tocopherol and alpha-tocopheryl succinate (FIG. 7). Results are shown as duplicate cultures (middle and bottom panel in each figure), with the control culture (no addition to culture medium) photographs presented in the top panel in each figure.

FIG. 8 is a graph illustrating apoptotic cell death as indicated by caspase activity, after six hour exposure to anoxia on porcine islets in the presence (Nutri) and absence (RM) of Vitamin E. Islets were pre-incubated for 24 h either in presence or absence of α+γ tocopherol (50 μM each) and then cultured for an additional 24 h under normoxia (95% Air, 5%CO2) or anoxia (95%N2, 5%CO2). Islets cultured under anoxia for 6 hrs have significantly increased caspase 3/7 activity while the combination of α+γ tocopherol blocks this increase.

FIG. 9, a series of photographs taken under microscopy, illustrates that porcine islets treated with a combination of α+γ-tocopherol exhibit a more normal morphology after exposure to anoxia than do islets treated with alpha-tocopherol or gamma-tocopherol alone. Islets treated with alpha- or gamma-tocopherol alone also exhibit a more normal morphology than do untreated islets exposed to anoxia. Porcine islets were hand picked and placed in wells (one islet/well in triplicates) in a 96 micro plate. The islets were pre-incubated either in regular porcine culture media (M199) or in media supplemented with alpha-tocopherol 100 μM, gamma-tocopherol 100 μM or the combination of alpha-+gamma-tocopherol, 50 μM each prior to exposure to anoxia. Islets were then incubated for additional 30 hrs either under normoxia (control—95% Air, 5%CO2) or anoxia (95%N2, 5%CO2) in the presence or absence of the indicated vitamin E concentration. At four different time points (0 h, 12 h, 24 h and 30 h), islets were removed from the incubator, examined microscopically and photomicrographs were obtained. Panel A: Porcine islets cultured with regular porcine culture media (M199) under anoxia. As indicated by the photomicrographs, anoxia caused a significant change in to the islet morphology. Panel B: Islets cultured with media (M199) supplemented with alpha-tocopherol (100 μM) under anoxia. Panel C: Islets cultured with media (M199) supplemented with gamma-tocopherol (100μM) under anoxia. Panel D: Islets cultured with media (M199) supplemented with a combination of alpha- and gamma-tocopherol (50 μM each) under anoxia. Strong protection of islets against anoxia damage is demonstrated by the combination of alpha- and gamma-tocopherol.

FIG. 10 is a graph indicating glucose levels measured in diabetic nude mice after transplant of 2000 islets cultured under anoxia for 6 hrs either in the presence (dashed line) or absence (solid line) of Vitamin E. Shaded background indicates glucose levels in the normal range. Five out of six mice transplanted with islets exposed to anoxia in the presence of Vitamin E cured whereas 1/6 exposed to the same condition in the absence of vitamin E failed to cure.

### DETAILED DESCRIPTION

The inventors have discovered that cell death can be prevented during any or all stages of the process involved in islet cell transplantation, including pancreas procurement, storage, and transportation; islet isolation; islet culture and transportation; and islet transplantation with an aqueous solution comprising an effective concentration of at least one solubilized Vitamin E homolog such as alpha-tocopherol, gamma-tocopherol, or a mixture of alpha- and gamma-tocopherol. Vitamin E homolog therapy may be provided to the pancreas donor (particularly where a partial transplant is planned and the donor will provide pancreatic tissue, but not an entire pancreas, or where xenotransplantation is contemplated), to the islet transplant recipient pre-transplantation, and/or to the islet transplant recipient post-transplantation. Vitamin E is generally considered to comprise eight different homologs: alpha-tocopherol, beta-tocopherol, gamma-tocopherol, delta-tocopherol, alpha-tocotrienol, beta-tocotrienol, gamma-tocotrienol, and delta-tocotrienol. Vitamin E homologs, as used herein, are compositions having the same general function as alpha-tocopherol (at levels of at least about 10 μM, and more preferably at least about 50 μM) and/or gamma-tocopherol in the present invention, and may also include, for example, derivatives and esters, such as succinate esters, of the tocopherols or tocotrienols. “Solubilized” Vitamin E homologs are those that have been admixed with a suitable solvent composition to form an aqueous solution. “Alpha-tocopherol, gamma-tocopherol, and combinations of alpha-tocopherol and gamma-tocopherol” are intended to include their functional equivalents from among the Vitamin E homologs, which include, for example, derivatives and esters of alpha-tocopherol, gamma-tocopherol, and combinations of alpha- and gamma-tocopherol.

One factor that contributes to cell death in transplanted islets is hypoxia. Pancreatic islets in vivo are highly vascularized, comprising only 2-3 percent of the total pancreatic mass, but receiving up to 15 percent of the pancreatic blood flow. The oxygen tension measured in pancreatic islets in vivo is almost double that in the exocrine pancreas. Because pancreatic islet cell isolation involves enzymatic digestion and mechanical separation, the normal vasculature is destroyed. Furthermore, if transplanted by intraportal embolization of islets into the liver, the cells are surrounded by oxygen-depleted venous blood. Anoxia and reoxygenation injury therefore can reduce islet quality during procurement of the pancreas itself, during islet isolation and islet cell separation, during islet cell culture, and during and post-transplantation. The composition and method of the present invention provide increased islet cell survival when islet cells are exposed to anoxia and/or nutrient depletion in each of these circumstances.

A limited number of approved islet resource centers provide islets for transplantation. Storage and shipping time are therefore important factors in islet cell survival. The method of the present invention provides increased survival of islets and cells during this procedure, ultimately resulting in a greater opportunity to produce successful transplant and glucose regulation in recipients.

Vitamin E homologs provide a protective benefit to isolated islet cells when present in the culture medium at a concentration of from about 1 μM to about 5 mM (and more preferably about 50 μM to about 500 μM). Where alpha-tocopherol is used, the level should preferably be at least about 10 μM, and more preferably at least about 50 μM. The inventors have demonstrated that gamma-tocopherol has a more protective benefit for preventing cell death in isolated islet cells than does alpha-tocopherol, and a combination of alpha- and gamma-tocopherol confers greater protection upon isolated islet cells than does either alpha-tocopherol or gamma-tocopherol alone. Excellent results were obtained when islet cells were cultured in medium containing 50 μM each of alpha- and gamma-tocopherol, for example. Derivatives of the Vitamin E homologs may be protective at lower concentrations. For example, alpha-tocopheryl succinate demonstrates a protective effect at concentrations of about 1 to about 5 μM and may be toxic to cells at much higher concentrations, while alpha-tocopherol's protective effect is exhibited at concentrations of at least about 10 μM, with an increased benefit noted at levels of at least about 50 μM.

To form an aqueous solution, the at least one Vitamin E homolog is dissolved in a suitable solvent such as, for example, an alcohol or d-α-tocopheryl polyethylene glycol 1000 succinate (Vitamin E TPGS, Eastman Chemical, Kingsport, Tenn.). Suitable alcohols include, for example, ethanol. A variety of solvents which are non-toxic to islet cells when combined in solution with one or more Vitamin E homologs and known to those of skill in the art may be used to solubilize the Vitamin E homolog. To facilitate contact between the islet cells and one or more Vitamin E homologs, an aqueous solution comprising one or more Vitamin E homologs may be added to islet cell culture medium before islet cells are added to the medium, after islet cells are added to the medium, or simultaneously with the addition of islet cells to medium or medium to isolated islet cells.

In one embodiment, a composition comprising a cell culture medium is provided by forming a water-miscible tocopherol solution comprising alpha-tocopherol and/or gamma-tocopherol in a solvent; adding the water-miscible tocopherol solution to an aqueous cell culture medium to provide an aqueous tocopherol medium comprising an about 1 μM to about 5 μM, and more preferably about 50 μM to about 500 μM) dispersion of gamma-tocopherol and/or alpha-tocopherol, or at least about 10 μM alpha-tocopherol, and more preferably at least about 50 μM, if not used in combination with another Vitamin E homolog.

Compositions of the present invention include isolation, transport and/or culture media comprising concentrations of from about 1 μM to about 5 mM of at least one Vitamin E homolog, and more preferably at least about 50 μM to about 500 μM of alpha-tocopherol, gamma-tocopherol, or a combination of alpha- and gamma-tocopherol. Suitable culture media are commercially available from a variety of sources (Invitrogen, Cambrex, Mediatech), and generally include suitable buffers, enzymes, and nutrients for maintenance of cells in vitro. Media ingredients may include, for example, essential and non-essential amino acids, calcium chloride, magnesium sulfate, sodium acetate, sodium phosphate, zinc sulfate, potassium chloride, sodium chloride, ascorbic acid, choline chloride, nicotinic acid, nicotinamide, and fatty acids such as, for example, linoleic acid.

Compositions of the present invention may be provided as kits, comprising liquid ready-to-use media or liquid or powdered media for reconstitution with sterile water, along with one or more separate vials of aqueous formulations of one or more Vitamin E homologs. Compositions may also comprise media containing Vitamin E homologs in conjunction with other ingredients that provide nutrients, an energy source, buffers, and other factors necessary for cell survival and maintenance during isolation, transport, and/or culture.

Kits may be provided for treatment of donor or recipient, the kits comprising aliquots or oral dosage formulations, such as capsules, geltabs, and the like, to provide an effective amount of at least one Vitamin E homolog, such as, for example, a combination of gamma- and alpha-tocopherol, via oral, intravenous, intraperitoneal, or other routes of administration. Such kits may provide sufficient dosage units to provide treatment for a number of weeks prior to transplantation. Preferably, such treatment will be provided for at least about 2 weeks and more preferably at least about 6 weeks.

The method of the present invention comprises contacting islets or isolated islet cells with a solubilized form of at least one Vitamin E homolog, preferably comprising alpha-tocopherol (to provide a concentration of at least about 10 μM and more preferably at least about 50 μM when added to media), gamma-tocopherol, or a combination of alpha- and gamma-tocopherol. The step of contacting islets or isolated islet cells may be performed while the islets reside within the donor, as the islets or islet cells are isolated (i.e., during the isolation procedure) or transported, during culture of isolated islets and/or islet cells, and/or during and following transplantation in a recipient.

Cells may be more easily contacted with an appropriate concentration of tocopherol as provided by the method of the invention during cell culture by incubating cells in a culture medium containing an effective concentration of a solubilized tocopherol. After transplantation, cells may be more easily contacted with an effective concentration of tocopherol by oral administration of tocopherol. More preferably tocopherol administration may be achieved by providing alpha-tocopherol, gamma-tocopherol, or a combination of alpha- and gamma-tocopherol as a modified release (i.e., sustained release) composition in conjunction with the cells during transplantation.

Microspheres are controlled-release, or modified-release, devices that have been used to encapsulate pharmaceutical compositions, nutrients, and other agents. Typically polymers suitable for use in microspheres and nanospheres are biocompatible, biodegradable and may be formed into microspheres or nanospheres by single or double emulsion techniques which are known in the art. Such polymers include poly(lactic acid), poly(lactide), poly(lactic-co-glycolic acid) and copolymers of lactic acid, phosphates such as ethyl phosphate, propylene oxide and other related copolymers which are suitable for uptake of small biologically active substances. Microspheres may be formed of poly(phosphoester-co-lactic acid) copolymers such as those recited in U.S. Pat. No. 6,166,173 and U.S. Pat. No. 6,805,876. Microparticles or nanoparticles may have one or more repeat units selected from phosphate, lactic acid, lactide, lactone, poly(ethylene oxide), and poly(propylene oxide), and may comprise at least one biocompatible polymer comprising a poly(phosphoester)-poly(D, L-lactide-co-ethylphosphate) copolymer.

Microcapsules may be provided to enclose both islets and Vitamin E homologs. Such microcapsules, microspheres, or other compositions may be, for example, from about 100 μm to about 250 μm. Matrices may be provided to enclose islets and to form a depot for microspheres, microcapsules, or other sustained- or modified-release compositions which further form one or more depots for at least one Vitamin E homolog. Microcapsules, microspheres, or other depots from which one or more Vitamin E homologs may be released to contact the surrounding cells within the islet may also be formed in a size so that they can be taken up into the islets to come in closer contact with cells within the intact islet. To facilitate absorption of microparticles into the vasculature of the islets, the diameter of the microparticles may be from about 2 to about 20 microns, and more preferably from about 4 to about 10 microns in diameter. The desired period for release is preferably at least about 3 days, with periods of at least about 10 days to at least about 30 days being of even greater benefit.

Additional antioxidant compositions may be added to compositions described by the present invention. Such antioxidant compositions may include, for example, complexes of tocopherols and tocotrienols with selenium; acetate, linoleate, and phosphate esters; butylates hydroxytoluene; butylated hydroxyanisole; propyl gallate; dodecylgallate; tert-butylhydroquinone; ethoxyquin; probucol; vitamin A as retinal, retinoic acid, and retinyl acetate; carotenes (alpha, beta, and gamma carotene, astaxanthin, lutein, lycopene, zeaxanthin); ascorbic acid and its calcium or sodium salts and ascorbyl palmitate; Coenzyme Q10 and other ubiquinols; glutathione, superoxide dismutase and their cofactors (e.g., selenium, selenomethane, manganese, copper); alpha-lipoic acid; dihydrolipoic acid; amino acids having antioxidant activity, such as cysteine and N-acetylcysteine; phytochemicals having antioxidant properties, such as isoflavonoids, diadzin, genistein, quercetin, morin, curcuma, apigenin, sesamol, chlorogenic acid, fisetin, ellagic acid, quillaia saponin, capsaicin, gisenoside, silymarin, kaempferol, ginkgetin, bilogetin, isogingetin, isorhamnetin, herbimycin, rutin, bromelain, levendustin A, and erbistatin. Compositions may also comprise other vitamins and nutrients such as B vitamins (e.g., folic acid and biotin), vitamin D and its derivatives and analogs, vitamin K, amino acids (and more preferably sulfur-containing amino acids), fatty acids, sugars, xarnitine and/or acetyl-L-carnitine. Any of the abovementioned nutrients may be added to compositions of the present invention, alone or in combination with other ingredients conferring increased islet cell health and survival, to promote islet cell survival in the method of the present invention.

Compositions and methods as described by the present invention may also be used to improve survival rates in other transplanted and transplantable cells, such as mesenchymal stem cells, hematopoietic stem cells, and bone marrow stem cells. Such cells may be contacted in the method of the present invention with at least one Vitamin E homolog in a similar manner to that used for islet cells, such as during cell culture, or post-transplantation by means of modified-release compositions comprising at least one Vitamin E homolog such as alpha-tocopherol, gamma-tocopherol, or a combination of alpha- and gamma-tocopherol.

The invention may be further described by means of the following non-limiting examples.

### EXAMPLES

Culture of Isolated Islets With Solubilized Tocopherol

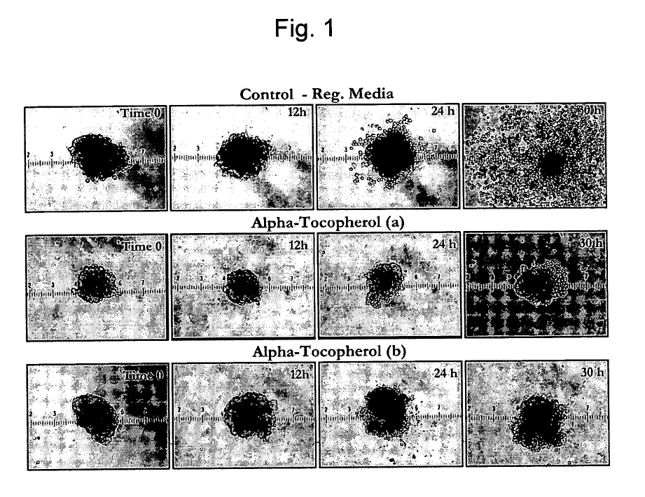

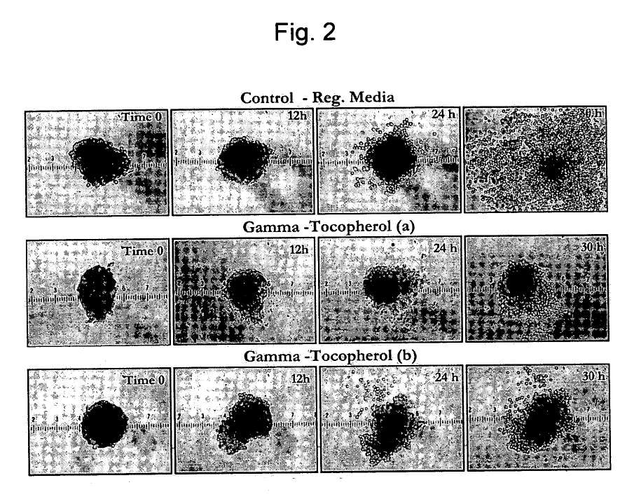

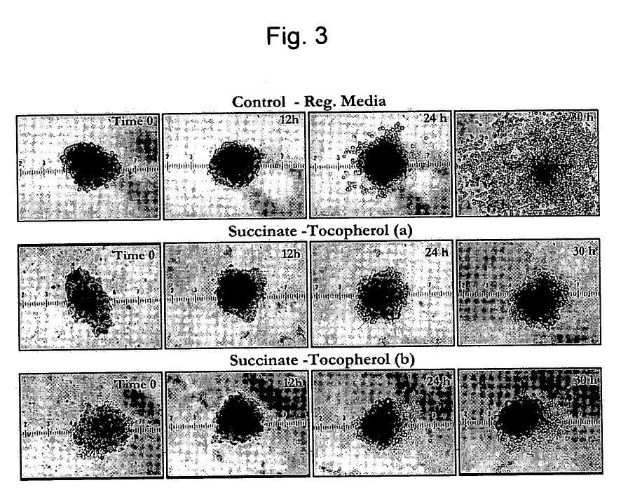

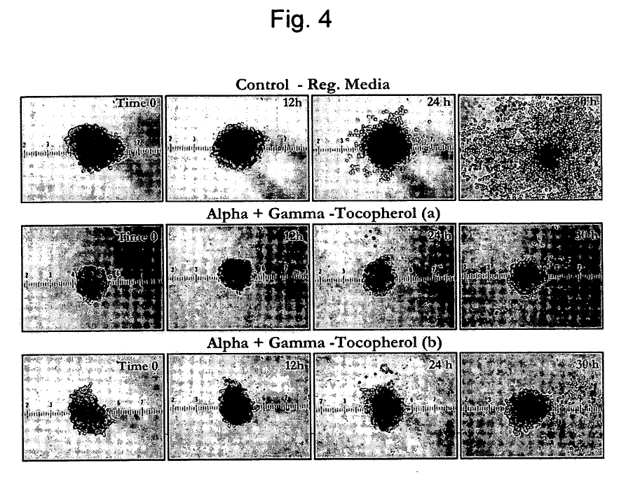

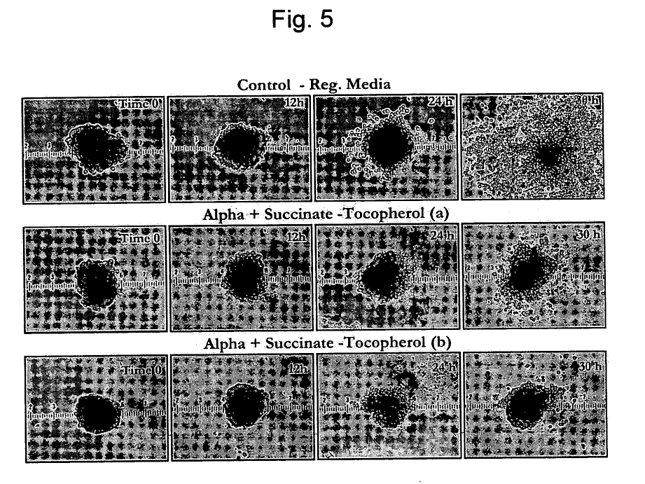

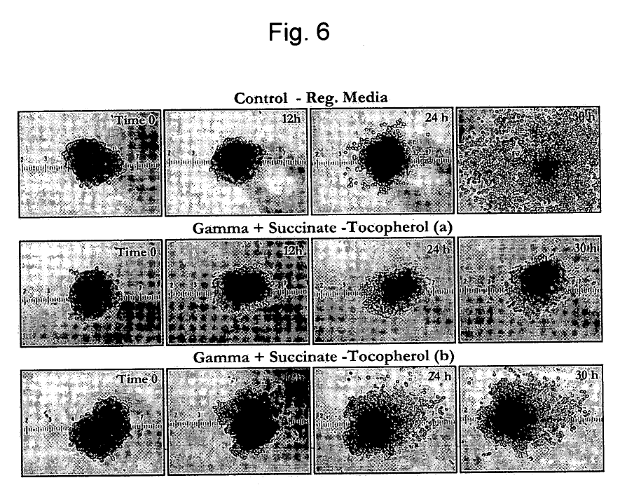

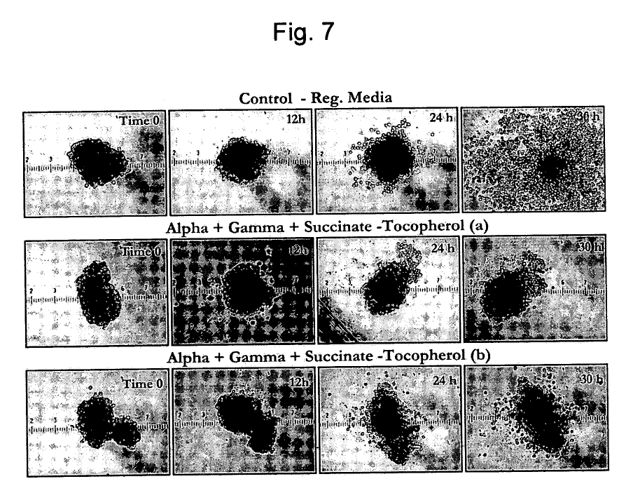

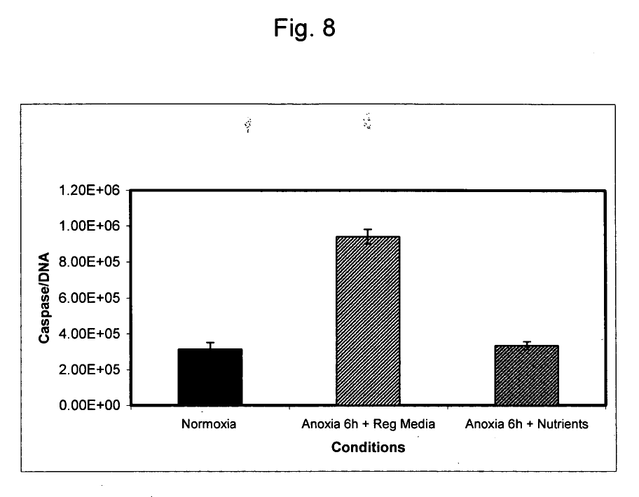

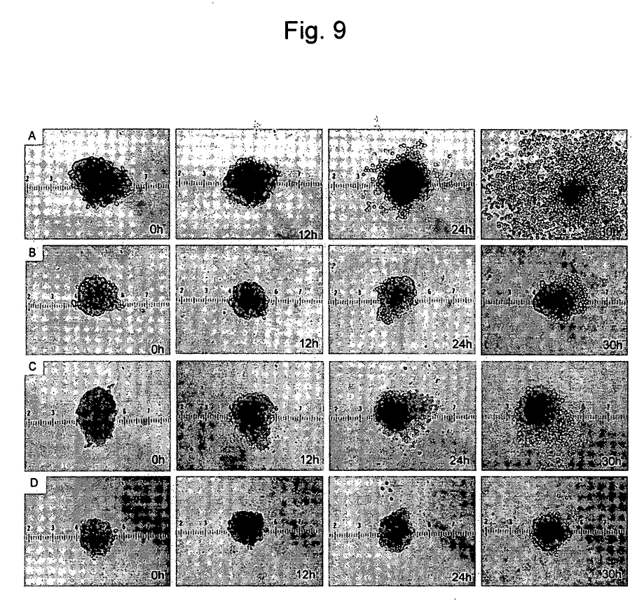

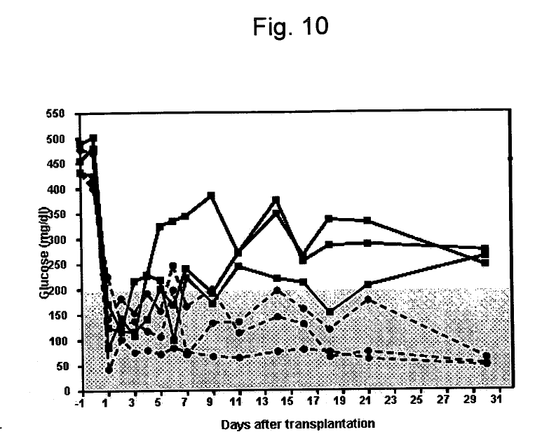

Single islets were hand-picked from a Petri dish under microscopy and transferred to wells in a 96-well microculture plate. Micronutrients comprising alpha-tocopherol (50 μM), gamma-tocopherol (50 μM), and succinate (5 μM) were added to wells in various combinations, with regular media (no added micronutrients) used as control. Islet cell survival was indicated by maintenance of a more compact cell mass, with disruption of the cell mass indicating islet cell death detected by microscopic means. Results are shown in FIG. 1 through FIG. 7. Results are provided as photographs of microscopic evaluation at each of the indicated timepoints after islets were introduced into medium containing each experimental treatment. Micronutrients added to each sample pictured in FIG. 1 through FIG. 7 are indicated in Table 1. Samples are shown in duplicate (a—middle panel, and b—bottom panel), with the corresponding control in the top panel.

TABLE 1
Micronutrient Treatment of Cultured Islets

Alpha-tocopherol
Gamma-tocopherol
Alpha-tocopheryl succinate
Alpha- + Gamma-tocopherol
Alpha-tocopherol + Alpha-tocopheryl succinate
Gamma-tocopherol + Alpha-tocopheryl succinate
Alpha-tocopherol + Gamma-tocopherol + Alpha-
tocopheryl succinate

Assessment of Islet Quality Following Exposure to Anoxia

Porcine islets were isolated and then cultured for 48 hours according to standard protocols Islets were then cultured for an additional 18-24 hours in media in the presence or absence of alpha-tocopherol and gamma-tocopherol (50 μM each). Islets from each group were cultured in their respective media for an additional 24 h exposed to a gas phase of either 95% air/5% CO₂ (control) or 95% N₂/5% CO₂ (anoxia). Islet quality was assessed at time intervals ranging from 6 to 24 hours from the initiation of exposure to anoxia with Oxygen Consumption Rate (OCR), ATP, Caspase 3/7 activity measurements (all normalized to DNA), microphotography and transplantation into diabetic nude mice. Data is reported as means±standard deviation for triplicate measurements.

OCR/DNA measurements demonstrated that 6 hours exposure to anoxia was sufficient to reduce islet viability to 44%±13% of control. Extending anoxic exposure to 24 hours further reduced viability to 35%±1% of control and resulted in severe islet- disintegration (single cells and small aggregates). Supplementation with alpha- and gamma-tocopherol enabled maintenance of viability to 91%±9% and 72%±4% of control at 6 and 24 hours, respectively, and prevented disintegration. ATP and Caspase activity measurements indicated trends consistent with the OCR measurements. Loss of islet viability under anoxia as well as protection by supplementation was confirmed by transplantation in nude mice. Similar results were obtained in experiments with human islets.

1. An islet cell culture medium comprising at least about 50 μM alpha-tocopherol.

2. An islet cell culture medium comprising at least one Vitamin E homolog chosen from the group consisting of gamma-tocopherol and a combination of alpha- and gamma-tocopherol, the at least one Vitamin E homolog being present at a concentration of from about 1μM to about 5 mM.

3. The culture medium of claim 2 wherein the at least one Vitamin E homolog is present at a concentration of from about 50 μM to about 500 μM.

4. A method for increasing islet cell survival when islet cells are exposed to anoxia or nutrient depletion, the method comprising contacting an islet cell with an effective amount of at least one Vitamin E homolog.

5. The method of claim 4 wherein the at least one Vitamin E homolog comprises alpha-tocopherol, gamma-tocopherol, or a combination of alpha- and gamma-tocopherol.

6. The method of claim 4 wherein the effective amount of the at least one Vitamin E homolog comprises about 1μM to about 5 mM of gamma-tocopherol or a combination of alpha-tocopherol and gamma-tocopherol.

7. The method of claim 6 wherein the effective amount of the at least one Vitamin E homolog comprises about 50 μM to about 500 μM of gamma-tocopherol or a combination of alpha-tocopherol and gamma-tocopherol.

8. The method of claim 5 wherein the effective amount of the at least one Vitamin E homolog comprises at least about 10 μM alpha-tocopherol.

9. The method of claim 5 wherein the effective amount of the at least one Vitamin E homolog comprises at least about 50 μM alpha-tocopherol.

10. The method of claim 4 wherein the step of contacting an islet cell with an effective amount of at least one Vitamin E homolog comprises contacting the islet cell with at least one Vitamin E homolog during islet isolation.

11. The method of claim 4 wherein the step of contacting an islet cell with an effective amount of at least one Vitamin E homolog comprises contacting the islet cell with at least one Vitamin E homolog during islet cell culture.

12. The method of claim 4 wherein the step of contacting an islet cell with an effective amount of at least one Vitamin E homolog comprises contacting the islet cell with at least one Vitamin E homolog during transplantation.

13. The method of claim 4 wherein the step of contacting an islet cell with an effective amount of at least one Vitamin E homolog comprises contacting the islet cell with at least one Vitamin E homolog post-transplantation.

14. The method of claim 13 wherein the step of contacting the islet cell with at least one Vitamin E homolog post-transplantation comprises administering one or more sustained-release forms of alpha-tocopherol, gamma-tocopherol, or a combination of alpha- and gamma-tocopherol in conjunction with islet cell transplantation.

15. The method of claim 4 wherein the step of contacting an islet cell with an effective amount of at least one Vitamin E homolog comprises contacting the islet cell with at least one Vitamin E homolog during islet transport.

16. A medicament for pretreatment of an islet cell transplant donor or recipient comprising a therapeutically effective dose of at least one Vitamin E homolog.

17. The medicament of claim 17 wherein the at least one Vitamin E homolog is chosen from among the group consisting of alpha-tocopherol, gamma-tocopherol, or a combination of alpha-tocopherol and gamma-tocopherol.
